(19) United States
(12) Patent Application Publication
(10) Pub. No.: US 2007/0172813 A1
(43) Pub. Date: Jul. 26, 2007

(54) Method for Evaluating Drug Candidates

(75) Inventors: Mary A. Reppy, Wilmington, DE (US); Charles F. Saller, Escondido, CA (US)

Correspondence Address: CONNOLLY BOVE LODGE & HUTZ LLP, P.O. BOX 2207, WILMINGTON, DE, 19899-2207, US

(73) Assignee: ANALYTICAL BIOLOGICAL SERVICES, INC., Wilmington, DE (US)

(21) Appl. No.: 11/161,281

(22) Filed: Jul. 28, 2005

Related U.S. Application Data
(63) Continuation-in-part of application No. 10/420,807, filed on Apr. 23, 2003.
(60) Provisional application No. 60/374,515, filed on Apr. 23, 2002.

(51) Int. Cl. C12Q 1/00

(52) U.S. Cl. 435/4

(57) ABSTRACT

Two-dimensional and/or three-dimensional polymeric or extended solid arrays comprising a polydiacetylene backbone, are used to evaluate the organic/water partition coefficient and/or oral absorptivity and/or ability of a compound to diffuse into cell membranes and/or transcellular permeability properties of a compound and/or the ionization state of a compound and/or the volume of distribution of a compound and/or the distribution of a compound into different tissues and/or the partitioning of a compound into cell organelles by monitoring the change in the fluorescence or phosphorescence upon exposure to the compound and optionally comparing it to a known change in fluorescence or phosphorescence, respectively. The method can also be used to evaluate the ability of a compound to bind to a protein.

### CROSS REFERENCE TO RELATED APPLICATIONS

This Application is a Continuation-in-part of application Ser. No. 10/420,807 filed on Apr. 23, 2003. Application Ser. No. 10/420,807 claims the benefit of U.S. Provisional Application 60/374,515 filed on Apr. 23, 2002.

### BACKGROUND OF THE INVENTION

The present disclosure relates to a method for evaluating properties of possible drug candidates in the drug discovery process, using two and/or three-dimensional polydiacetylene arrays or extended solid polydiacetylene arrays. The arrays are exposed to the drug candidates and the fluorescence or phosphorescence of the arrays measured. The extent of emission of arrays exposed to drug candidates can be used to estimate and/or predict the organic/water partition coefficient of the drug candidates, and/or their oral absorption and/or their ability to diffuse into cell membranes and/or their transcellular permeability and/or compound ionization state, and/or the volume of distribution of the compounds and/or their distribution into different tissues and/or their partitioning into cell organelles. The arrays can also be used in assays that evaluate the binding of drug candidates and other compounds to proteins, such as human blood plasma proteins or other biological molecules.

Polydiacetylenes are conjugated polymers with backbones of alternating double and triple bonds formed from the 1,4-addition polymerization of 1,3-diacetylenes. Polydiacetylenes generally absorb well in the visible region of the spectrum, and hence are highly colored, ranging from blue to yellow. There has been intense interest in the non-linear optic properties of polydiacetylenes and extensive study has been made of both the solvo-chromic properties of solubilized polydiacetylenes and the thermo-chromic properties of polydiacetylene films and single crystals. It is well known that to form polydiacetylene, the diacetylene monomers must be in an ordered packing to allow the polymerization to occur. It seems to be generally accepted, though the inventors are not bound herein, that the packing of the side chains can affect the conjugation length of the backbone, and hence the chromic and emissive properties.

Diacetylene monomers have been used to form various ordered systems, including crystals, liquid crystals, liposomes and films that were then polymerized to form the polymer. Liposomes have been made from monomers with two diacetylene chains and polar head groups (such as phosphotidylcholines, and its analogues) and from monomers with single diacetylene chains. The liposomes can be polymerized with UV light or gamma-radiation. Monomer films have been formed by Langmuir Blodgett methods or cast from solvents and then also polymerized with UV light or gamma-radiation. The choice of monomer structure, conditions of liposome or film formation, and polymerization conditions all affect the conjugation length of the polydiacetylene backbone, and hence the color of the system. Upon heating, these polymerized systems can undergo a change in the effective conjugation length, from the longer length forms (blue and purple) to the shorter length forms (red and yellow). In the case of the packed polymer arrays that form the films and liposomes, it is generally accepted that changes in the environment that affect the organization and packing of the side chains coming off the conjugated backbone can affect the conjugation length and hence the chromic and electronic properties of the polymer.

These polydiacetylene films and liposomes have been suggested for chromogenic assays that depend upon color change (Charych et al, U.S. Pat. No. 6,001,556; Charych et al, U.S. Pat. No. 6,180,135; Charych et al, U.S. Pat. No. 6,080,423; Charych, U.S. Pat. No. 6,183,772; Charych et al, U.S. Pat. No. 6,022,748).

Color is an absorbance property; the colors we see are related to the wavelengths of light that the species is absorbing. For example if the species absorbs light primarily at 650 nm, we will see it as blue, while if it absorbs primarily at 550 nm, we will see it as red. Color arises from absorbance of light in the visible range. Most colored species are not fluorescent. If a colored species is fluorescent, it will normally appear one color, but when it is excited with the appropriate wavelength, it will glow with the color of the emitted light. For example, a fluorophore may look like an orange powder, but glow green under a UV lamp.

Polydiacetylenes can show fluorescence. However, their ability to fluoresce is dependent on the structural form and organization of the polymers (particularly the conjugation length and polymer chain aggregation state), whether in solution, a film, or formed into liposomes or other three-dimensional structures.

It is known that polydiacetylene films have an intrinsic fluorescence when produced in the red or yellow form, and are not fluorescent (by conventional measurements) when the film is made in the blue form (Yasuda A. et al, Chem. Phys. Lett., 1993, 209(3), 281-286). This fluorescent property of the films has been used for microscopic imagining of film domains and defects.

More recently, it has been discovered that the change in polydiacetylene arrays from a non-fluorescent to a fluorescent state can be used for detecting an analyte by measuring the emission of an array incorporating a ligand, receptor or substrate for the analyte. Furthermore the extent of this change can be magnified by incorporation of suitable fluorophores. These discoveries are described in patent applications PCT Patent Application WO/00171317, and U.S. patent application Ser. No. 09/811,538 to Reppy et al., entitled “Method for detecting an Analyte by Fluorescence”, and assigned to Analytical Biological Service Inc, the assignee of this application, disclosures of which are incorporated herein by reference.

### SUMMARY OF THE INVENTION

The present disclosure provides a method to evaluate properties of compounds by measuring the effect of the compounds on the fluorescence or phosphorescence of two-dimensional and/or three-dimensional polymeric or extended solid arrays comprising a polydiacetylene backbone. More particularly, the present disclosure provides for the evaluation of compounds considered to be potential drug candidates, which comprises contacting the solution of the compound to be tested with a two-dimensional and/or three-dimensional array comprising a polydiacetylene backbone. The change in fluorescence or phosphorescence is measured or detected. The change and it's magnitude can be used to estimate the organic/water partition coefficient and lipophilicity and/or the likely oral absorption of the compound, and/or their ability to diffuse into cell membranes and/or their transcellular permeability, and to evaluate the compound as a candidate for oral administration. This change can also be used to estimate the ionization state of a compound, and/or the volume of distribution of a compound and/or its distribution into different tissues and/or its partitioning into cell organelles. The method can also be used to assess the binding of compounds to proteins or other macromolecules. The method can be used to rapidly screen thousands of compounds in an automated fashion in the drug discovery process. Moreover, the change in fluorescence or phosphorescence can optionally be compared to the change in fluorescence or phosphorescence, respectively, of the same arrays exposed to standard or reference compounds in solution.

Other objectives and advantages of the present disclosure will become readily apparent to those skilled in the art from the following detailed description, wherein only the preferred embodiments are shown and described, simply by way of illustration of the best mode contemplated of carrying out the disclosure. As will be realized, the disclosure is capable of other and different embodiments, and its several details are capable of modification in various apparent respects, without departing from the disclosure. Accordingly, the drawings and description are to be regarded as illustrative in nature and not as restrictive.

### BRIEF DESCRIPTION OF THE DRAWINGS

FIGS. 1A, 1B and 1C illustrate a sequence of employing both a two dimensional array and a three dimensional array in a process according to the present invention.

FIGS. 2A and 2B, respectively show plots of change in emission between an initial reading and a final reading versus Log (P) (FIG. 2A) and oral absorptivity (FIG. 2B) for coatings exposed to test compounds.

FIG. 3 is a plot of the Log (P) versus liposome emission for test compounds.

FIGS. 4A and 4B, respectively show plots of change in emission versus oral absorptivity graphs for liposomes with a test compound set (FIG. 4A) and with only amine test compounds from the set (FIG. 4B).

FIG. 5 is a plot of the slope of change in emission over time of liposomes exposed to compounds plotted versus the % oral absorptivity of the compounds.

### DETAILED DESCRIPTION

In order to facilitate an understanding of the present disclosure the following definitions which are used herein are presented:

Test compound: a chemical compound added to the test materials.

Partition coefficient: ratio of the concentrations of a compound in equilibrium between two solvent phases, usually octanol and water or buffer. The concentration of the compound in the organic phase is the numerator of the ratio.

Log(P): the logarithm (base 10) of the partition coefficient in octanol/water. This definition includes what are commonly referred to as “log(D)” values; i.e., partition coefficients of ionic compounds in octanol/water measured at various pH's.

Oral absorptivity: portion of orally administered material that is found in the blood stream.

Volume of distribution: a measure of the partitioning of a compound between plasma and all tissues combined

Tissue uptake: a measure of the partitioning of a compound into a specific tissue. Tissue refers to both tissues and organs.

In addition to facilitate an understanding of the present disclosure, the discussion that follows will be predominantly directed to the fluorescent aspect of the present disclosure with the understanding, as will be apparent to those skilled in the art, that the discussion likewise applies to the phosphorescent aspect.

The preferred two-dimensional and three-dimensional arrays employed according to the present disclosure comprise a polydiacetylene backbone. The arrays are prepared by polymerization of precursor diacetylene arrays. The diacetylene precursor two and three-dimensional arrays may also contain species that are not diacetylenes.

The polydiacetylene backbones employed according to the present disclosure are known and need not be described herein in any detail and can range from being oligiomeric (from the reaction of three or more monomers) to polymeric. For example see U.S. Pat. No. 6,001,556 to Charych et al, disclosure of which is incorporated herein by reference.

In this embodiment the polydiacetylene is formed from polymerizing a three-dimensional or two-dimensional array of diacetylenes. The array may also contain non-diacetylene species such as natural and unnatural phospholipids, cholesterol, steroids, gangliosides, sugars, lipids, proteins and other species including any of those found in cell membranes to increase the biomimetic character of the array. The array may also contain other non-diacetylene species and multiple diacetylene species.

Also, side chains with ordering head groups are typically bound to the polydiacetylene backbone. The head groups are typically polar. The polydiacetylene amphiphilic side chains also add to the biomimetic character of the array.

The preferred arrays are formed by polymerizing arrays of diacetylene monomers. The typical monomers are single or multi-tailed diacetylene surfactants with polar head groups. More typically used are single or bis-tailed diacetylene surfactants with polar head groups. The disclosure is not dependant on the use of any specific diacetylene surfactant, tail structure, or polar head group, but can be used with any diacetylene monomer that can be polymerized to give polydiacetylene in its non-fluorescent form or polydiacetylene in a fluorescent form that can converted to another fluorescent form with a different magnitude of emission.

Materials typically used as head groups in the present disclosure include, but are not limited to: carboxylic acids, carboxylate salts, amides, ethanol amide, amines, ammoniums, imines, imides, alcohols, carbamates, carbonates, thio-carbamates, hydrazides, hydrazones, phosphates, phosphonates, phosphoniums, thiols, sugars, sulfates, sulfonates, sulfonic acids, sulfonic amines, sulfonamides, amino acids (including glutamate and glutamine), peptides, nitro-functionalized moieties, carbohydrates, choline, ethylene glycol, oligiomeric ethylene glycol, poly(ethylene glycol), propylene glycol, oligiomeric propylene glycol, and poly(propylene glycol), and combinations thereof.

When the arrays of the present disclosure are to be secured or anchored to a support surface, the tails or head groups of the lipids can be selected to provide this function.

The two-dimensional and three-dimensional arrays of the present disclosure can be produced in any number of forms. Liposomes are one of the suitable three-dimensional array forms that can be produced. The liposomes can be formed in a number of different sizes and types. For instance, it is possible to form the liposomes as simple bi-layer structures. Additionally, they can be multi-layered in an onion type structure. Their size can also be varied. A suitable two-dimensional array form that can be produced is a film. The film can be mono-layered, bi-layered, or multi-layered.

Numerous other shapes can also be produced. Lamellae (Rhodes et al, Langmuir, 1994, 10, 267-275), hollow tubules and braids (Frankel et al, J. Am. Chem. Soc., 1994, 116, 10057-10069.), ribbons, crystals, lyotropic and thermotropic liquid crystalline phases, gels and amorphous structures are among the other shapes that can be formed. Three-dimensional shapes collectively may be in colloidal suspensions or solutions. When these assemblies are immobilized they can collectively form even larger constructs.

To facilitate a further understanding of the present disclosure, the preferred diacetylene arrays are discussed below, but it is understood that the various structures discussed can employ other arrays.

Diacetylene liposomes can be converted to tubules before polymerization by controlled cooling, concentration changes, or addition of ethanol. Tubules can also form with dispersion of diacetylene amphiphiles into aqueous solution. The tubules can be photopolymerized to give either the non-fluorescing form or the fluorescing form of polydiacetylene; either form can be used in assays with fluorescence monitoring. Polydiacetylene can also be formed as blue or red gels with a net-work structure of aggregated fibers. Polydiacetylenes can be used in the formation of composite materials, including layering with inorganic clays. Film arrays of diacetylenes or polydiacetylenes may be used in the free standing form, or supported on glass, ceramic, polymer, paper, metal, or other surfaces. The supports may be porous, including, but not limited to, nano and micro porous membranes. Diacetylene coatings may also be cast onto glass, ceramic polymer, paper, metal or other surfaces and photopolymerized to give the polydiacetylene array described above.

Diacetylene and polydiacetylene three-dimensional and two-dimensional arrays may be attached to, supported on, or absorbed in, solids, including, but not limited to: polymers such as polystyrene, polycarbonate, polyethylene, polypropylene, cellulose, cellulose esters, nylon and polyfluorocarbons such as Teflon®(polymers of tetrafluoroethylene), perfluorinated ethylene-propylene copolymers, copolymers of tetrafluoroethylene and perfluoroalkoxy, copolymers of tetrafluoroethylene and ethylene, polymers of chlorotrifluoroethylene, and copolymers of chlorotrifluoroethylene and ethylene, polyvinylene difluoride, silicon chips, beads, filters and membranes, glass, gold, silica, sephadex, sepharose, porous or swelling solids such as polyacrylates and polyacetonitrile, and sol-gels. The solids listed here may also be further modified by chemical, or plasma gas, or other treatment. In the case of diacetylene three-dimensional and two-dimensional arrays, they are polymerized after incorporation with or attachment to the solid support.

An embodiment of solid supported polydiacetylenes is as an array on nano-porous membranes. Diacetylene liposomes, or other three-dimensional arrays, can be forced in and onto membranes including 100, 200 and 400 nm membranes and photopolymerized to create a polydiacetylene coating. The three-dimensional arrays may fuse together. These coated membranes are stable at room temperature, in air, and exposed to light, for at least 12 months. The polydiacetylene array coating exhibits some resistance to abrasion. The polydiacetylene arrays can be converted from the non-fluorescent to the fluorescent form or from one fluorescent form to another fluorescent form with a different magnitude of emission or the fluorescent to the non-fluorescent form in response to environmental changes including exposure to a solution containing a test compound. In other words, the fluorescence of the arrays can be either decreased or increased by exposure to the test compound.

Nanoporous membranes are available in many materials, including: alumina, polyfluorocarbons such as Teflon® (polymers of tetrafluoroethylene), perfluorinated ethylene-propylene copolymers, copolymers of tetrafluoroethylene and perfluoroalkoxy, copolymers of tetrafluoroethylene and ethylene, polymers of chlorotrifluoroethylene, and copolymers of chlorotrifluoroethylene and ethylene; nylon, polycarbonate, cellulose, cellulose esters, polyvinylene difluoride (PVDF), and glass and also in a variety of pore sizes. We envision using any of these membrane types with pore sizes typically up to about 600 nm for preparing solid supported polydiacetylenes. The membrane surfaces may be further modified by chemical, or plasma gas, or other, treatment. Microtiter plates are available and can be made with nanoporous membranes for the well bottoms that can be precoated with the diacetylene or polydiacetylene arrays or coated with the diacetylenes arrays in situ and polymerized.

By way of example, the diacetylene two-dimensional and three-dimensional structures are photopolymerized with UV light, or gamma-radiation, to give organized polydiacetylenes with the longer conjugation lengths characterized by absorption maximum in the range of 500-800 nm, preferably in the range 600-750 nm, and a blue to purple color. The photopolymerization results in creating mainly the non-fluorescing form and therefore exhibiting low overall fluorescence relative to the background. The term “non-fluorescent form” as used herein also refers to these polymers which have low overall fluorescence exhibiting a fluorescent signal above 500 nm that is only about 1-3 times that of the background and less than that of the corresponding fluorescent form. The term “emission” as used herein refers to the intensity of fluorescence emitted at a wavelength or over a range of wavelengths. Typically, the “non-fluorescent form” exhibits a fluorescent emission above 500 nm that is at least about 10% lower and more typically at least about 50% lower than that of the corresponding fluorescent form. Some diacetylene two-dimensional and three-dimensional arrays give polydiacetylene in the fluorescent forms upon photopolymerization; these may still be used in assays if interaction with a test compound converts the arrays to a fluorescing form having a different measurable emission that is either lower or higher than the original emission, or has the wavelengths and/or ratios of emission maxima intensities shifted. Arrays may also be heated or exposed to other environmental changes, for example, a change in pH, to convert them to the fluorescent form before use in assays. In an application described here, the exposure of polydiacetylene arrays to compounds of higher log(P) can lead to a drop in intensity of the measured emission of the array versus the emission of arrays exposed to compounds of lower log(P) and to reference solutions.

The polydiacetylene fluorescence of the fluorescent form may be excited by light with wavelengths between 300 and 600 nm, and consists of a broad fluorescence above 500 nm with one or two maxima though the invention is not bound by these specifics.

The conditions that cause conversion to the fluorescent form also may cause chromic changes that appear to the eye as a blue to red shift, and can be quantified by measuring the UV/VIS absorption spectrum. Change in fluorescence and change in absorbance at specific wavelengths do not necessarily linearly correlate. It seems likely, though the inventors are not bound herein, that a rise in the fluorescence emission can be a result of either a rise in the population of shorter conjugation length polydiacetylene backbones, or a decrease in the quenching longer conjugation length polydiacetylene backbones, or both. The relative change in fluorescence can be an order of magnitude or more, greater than the relative changes measured in the UV/VIS absorption spectrum upon this transformation. This means that fluorescence can provide a more sensitive measure of change in the liposomes than the direct chromic response. This increase in sensitivity makes the novel fluorescence detection method of use in many areas where calorimetric detection would simply not be sufficient. These include drug discovery assays. For films, the fluorescent/non-fluorescent properties of the polydiacetylenes can be used as a new detection method and would also provide increased sensitivity compared to calorimetric detection in immobilized sensing systems. Fluorescence detection also allows sensing platforms to use opaque supports, whereas calorimetric detection requires clear (e.g. quartz glass or UV/VIS-transparent plastic) supports.

Interaction of three-dimensional polydiacetylene arrays in suspension or solution may lead to coalescence and/or aggregation of the individual arrays. These phenomena may be triggered or caused by addition of other species to the three-dimensional polydiacetylene array suspensions or solutions. Coalescence and/or aggregation can lead to decreases in fluorescence emission of three-dimensional polydiacetylene arrays in suspension or solution; the drop in emission can be used to detect the coalescence and/or aggregation. The drop in emission used to determine a property of the added species, or for detection of its presence. Colorimetric methods are less suitable for determining coalescence and/or aggregation because the relative ratios of UV/VIS absorbance peaks do not necessarily change as the three-dimensional arrays come together, and the formation of aggregates in solution can lead to noisy absorbance signals.

It is possible to dilute the polymer array with other non-diacetylene surfactant species while still maintaining the liposome or film structure.

In a further embodiment of the disclosure, the arrays may incorporate other fluorescent or phosphorescent species. These fluorophores and phosphors may be organic, biological, inorganic or polymer compounds, complexes, or particles. The fluorophores or phosphors can enhance the magnitude of the change in the fluorescence or the phosphorescence of the polydiacetylene arrays as the arrays change from one fluorescent or phosphorescent form to another. The fluorescence or phosphorescence of the added fluorophores or phosphors can also be monitored during this conversion, either as an internal standard if the fluorophore's fluorescence or phosphors's phosphorescence are not affected by changes in the polydiacetylene, or as an additional measure of the conversion when the fluorophore's fluorescence or phosphors's phosphorescence does change. In addition certain fluorophores can undergo excited state energy transfer processes that change the overall fluorescence of the array, and increase the quantum yield.

Many of the suitable fluorophores are lipophilic and are expected to incorporate into the alkyl region of the arrays, while others are polar or charged and are expected to end up in the head group region/aqueous interface, or in the water solution. The change in the arrays' fluorescence and the added fluorophore's fluorescence have been monitored as the arrays changed from the non-fluorescing to a fluorescing form, and from a fluorescing form to a less fluorescent form, under the action of heat, changes in pH or other environmental changes, chemical reactions, or during analyte detection. Various fluorophore additives had the effect of enhancing the percentage change in the array fluorescence response upon changing from the non-fluorescing form to the fluorescing form and from a fluorescing form to a less fluorescent form. The fluorescence of the added fluorophore was also monitored and also indicated the change in the liposome form from non-fluorescing to fluorescing, with some fluorophores showing an increase in their fluorescence and others a decrease.

For instance, the added fluorophores may optically and/or electronically interact with the polydiacetylene polymer. There are several ways that the fluorophores and the array can optically/electronically interact, including but not limited to the following means:

(1) By the fluorophore absorbing the fluorescence of the array or by the array absorbing the fluorescence of the fluorophore.

(2) By the fluorophore absorbing the excitation light, becoming excited and then transferring energy from its excited state to the polydiacetylene array causing it to fluoresce. This process is known as Resonance Energy Transfer (RET) and also as Fluorescence Resonance Energy Transfer (FRET). This RET process allows the polydiacetylene fluorescence to have the time decay properties of the fluorophore.

(3) By the polydiacetylene array, in its fluorescent form, absorbing the excitation light and then transferring the energy from its excited state to the fluorophore leading to the fluorophore fluorescing. This RET process can lead to an increase in the effective Stokes shift of the system and also increase the overall quantum yield.

(4) By the excited state of the fluorophore transferring an electron to the array or by the excited state of the array transferring an electron to the fluorophore. This process is known as Photoinduced Electron Transfer (PET).

(5) By the array absorbing the excitation light needed for fluorophore fluorescence or the fluorophore absorbing the excitation light needed for array fluorescence.

(6) By the fluorophore quenching the fluorescence of the array.

(7) By the array quenching the fluorescence of the fluorophore.

Addition of fluorophores or phosphors to the polydiacetylene array can make it possible to increase the extent of the change in fluorescence or phosphorescence of the array during an assay, thus increasing assay sensitivity; and also to monitor the fluorescence of the fluorophore or phosphorescence of the phosphor during the assay as a second measure of change in the polydiacetylene array caused by the analyte. The RET interaction between the fluorophore and the polydiacetylene array is discussed below.

Fluorescence Resonance Energy Transfer (RET):

While fluorescence monitoring using the intrinsic fluorescence of polydiacetylene is an improvement over absorbance (i.e. calorimetric) monitoring, polydiacetylene is a relatively weak fluorophore. Fluorescent polymers are often poor fluorophores because the conjugated chains can act as excited state traps and provide mechanisms for non-radiative relaxation of the excited state, leading to quenching of the fluorescence (Warta R. et al, J. Chem. Phys., 1988,1, 95-99). It is theorized that the long conjugation lengths of blue polydiacetylene provide very effective trapping of the excited states which is why the blue form is effectively non-fluorescent (Morgan J. et al, Chem. Phys. Lett., 1992, 196, 455-461). Red and yellow polydiacetylene arrays may contain mixtures of polymer chains of different conjugation lengths, and so it is quite likely that inter-chain quenching as well as intra-chain quenching, reduces the fluorescence of the shorter conjugation lengths.

The overall fluorescence of the arrays can be increased by incorporating certain fluorophores in the lipid regions, at the lipid/water interface and in the water solution itself (Reppy, M. A. “Signal Generation from Switchable Polydiacetylene Fluorescence”, Mat. Res. Soc. Symp. Proc. 2002, 723, O5.9.1-O5.9.6). The RET process with certain fluorophores also significantly increases the Stokes shift from <100 nm to >200 nm. This is important when using plate readers to measure fluorescence as they often have less than optimal optics, or when reading the fluorescence of solids where there is usually high background from reflection of the excitation light off the surface. The incorporated fluorophores are directly excited by the wavelengths used to excite the polydiacetylene only to a minor extent or not at all, and so they do not make the non-fluorescent polydiacetylene arrays containing these fluorophores significantly fluorescent.

It is also possible for the fluorophores to transfer energy from their excited states to the fluorescent form of the array, causing it to fluoresce. A fluorophore can be excited at a wavelength that polydiacetylene is not excited by, and then transfer energy to the polydiacetylene leading to polydiacetylene fluorescence. This also may increase the effective Stokes shift of the overall polydiacetylene array. If the fluorophore has a long lifetime, for example certain terbium and europium compounds have long excited state decay times, the polydiacetylene fluorescence decay will have a similar lifetime. This makes it possible to perform time-resolved fluorescence (TRF) measurements using polydiacetylene arrays and common TRF readers (e.g., the Perkin Elmer/Wallach Victor2V). TRF has the advantage of giving a lower background as the background fluorescence usually has decayed before the measurement is made.

The fluorescence or phosphorescence can be read with any equipment known in the art for fluorescent or phosphorescent measurements, including, but not limited to, fluorometers with cuvette and fiber optic attachments, plate readers, hand held readers, fluorescence microscopes, CDC cameras, and by eye. This sensing method may be readily used in the multi-well plate formats of high-throughput screening.

The method of the present disclosure can be used in the field of drug discovery. Developers of pharmaceutical chemicals wish to test their compounds for biologically relevant physical properties such as solubility, lipophilicity, ionization state at different pHs (i.e., pKa), oral absorption, etc. In particular, developers are very interested in whether a drug, or potential drug, can be orally administered. If a drug can be orally administered it can be used much more widely and with much less expense than a corresponding drug that must be intravenously administered; hence the orally administered drug is likely to have a much larger market. Developers can evaluate the likelihood of their compounds having drug-like characteristics that would allow oral administration by using the so-called Lipinski rule of five. The Lipinski rule of five suggests that compounds with drug-like characteristics have fewer than five hydrogen bond donors, fewer than ten hydrogen bond acceptors, molecular weights lower than 500 g/mol, and partition coefficients (log(P)) less than five. The first three criteria can be evaluated from the structure of the compounds; the last (i.e., log (P)) must either be measured directly or estimated through other means.

Measuring the equilibrium concentrations of compounds between octanol and water and taking the logs of the equilibrium constants directly determines partition coefficients. The direct measurement of partition coefficients requires significant amounts of sample and an analytical method for quantitating the concentrations of the species in the two phases. These measurements are not suitable for high throughput screening. Compounds have also been partitioned between buffer and phospholipid liposomes (Balon K. et al., Pharmaceutical Research, 1999, 16(6), 882-888), this also requires a separate analytical step for measuring the compound concentrations. Phospholipid liposomes are usually not stable for extended periods and/or over wide pH ranges.

Animal and human studies to determine the oral absorption of compounds are expensive and time-consuming, and can only be used for a small number of compounds. A human intestinal derived cell line known as Caco-2 cells has been used for evaluation of compound oral absorptivity (Artursson P. and Karlsson J., Biochemical and Biophysical Research Communications, 1991, 175(3), 880-885). The cells are grown in a layer on filters (a process which takes over two weeks), test compound solutions in buffer are put over the cell layers and the permeability of the compounds through the cells is measured by analyzing (in a separate step) the concentration of the compound found on the other side of the cell layer. Currently it is not possible to efficiently evaluate large numbers of compounds (>50) in parallel with this method and the cells are not compatible with a wide pH range. Other cell lines have been used for similar testing. An alternative that uses 96-well filter plates, where the filters are impregnated with a mixture of hexadecane, has been proposed (Wohnsland F. and Faller B., Journal of Medicinal Chemistry, 2001, 44, 923-930); the passage of test compounds through the membrane is measured separately. Similarly, membrane supported lipid mixtures (PAMPA) are also used (Sugano K. et al., Journal of Biomolecular Screening, 2001, 6(3), 189-196). Silica beads coated with phospholipid bilayers (TRANSIL) have been used for assessing compound membrane affinity (Loidl-Stahlhofen A. et al, Journal of Pharmaceutical Sciences, 2001, 90(5), 599-606): the beads are incubated with compound solutions and then separated, the amount of compound remaining in solution is then quantitated by chromatography. Chromatographic methods have been proposed where chromatographic columns are packed with silica functionalized with phospholipid ligands (Ong S. et al, Journal of Chromatography A., 1996, 728, 113-128) and the retention times of compounds are roughly correlated with oral absorptivity; again these methods are expensive and impractical to use for high throughput screening of thousands of compounds.

In all of these prior methods it is necessary to add a separate step for detecting whether the compounds have partitioned into, or passed through, the cells, membranes etc, by testing the solutions. This has been achieved by radiolabelling of the compounds, measurement of the UV-Vis (absorbance) spectra of the solutions, chromatography and chromatography coupled with mass spectroscopy (LC/MS) and potentiometric measurements. Radiolabelling is expensive and makes the compounds hazardous to handle; it is not practical for screening large numbers of compounds. UV-Vis measurements require that the compound have a chromophore, which is not always the case, and also determination of where the compound has maximum absorbance. Chromatographic methods have low throughput; LC/MS in addition requires expensive equipment. Potentiometric measurements are technically demanding and require specialized equipment. The present disclosure makes possible a one step detection or evaluation method that works with a wide range of compounds, does not require the test compounds to have chromophores or radiolabels, and can be performed using commercial equipment that is readily available.

There is currently no experimental high-throughput screening (HTS) method for determining a compound's volume of distribution or uptake by different tissues. A drug compound's propensity to be taken up by different tissues affects whether it will reach its target site, whether it will cause undesirable side-effects and hence, whether it will be clinically viable.

Existing methods for predicting compound distribution in humans rely largely on animal studies; these studies are laborious to carry out and the results may not be applicable to humans. There are expensive screening techniques such as positron emission tomography (PET) and nuclear magnetic resonance (MRS) (REF: Langer O.; Müller M. “Methods to Assess Tissue-Specific Distribution and Metabolism of Drugs” Curr. Drug Metab., 2004, 5, 463-481) for screening compound distribution in humans. These methods require very expensive specialized equipment and synthesis of radio-labeled compounds. Microdosing experiments and clinical microdialysis have also been used in humans; microdosing experiments use compounds at levels that are not pharmacologically relevant so the results may not be clinically relevant, and also require either radio-labeled compounds or the use of accelerated mass spectroscopy. Clinical microdialysis is an invasive procedure. These methods require human subjects, which leads to a range of ethical complications. None of these methods are suitable for screening large number of compounds in a parallel fashion. A high-throughput in vitro method for predicting distribution of drug compounds in the human body is an important addition to pre-clinical screening of new chemical entities (NCE).

The method of the present disclosure makes it possible to predict (e.g., closely approximate) or estimate one or more of the compound physical properties of biological relevance of interest to drug manufacturers, in a more cost-effective manner; and is significantly better for screening large numbers of compounds as compared to prior methods. Accordingly, the method of the present disclosure makes it possible to estimate or predict these compound properties earlier in the drug development cycle. The method of present disclosure is suitable for high-throughput screening of thousands of compounds in an automated fashion and can be used to evaluate a wide range of compounds.

One embodiment of the testing method of the present disclosure involves adding test compounds in aqueous or DMSO solutions to solutions of polymerized three-dimensional arrays (e.g. liposomes or tubules) in a 384-well or 96-well microtiter plate and measuring the emission of the wells after an incubation time ranging from minutes to hours. The emissions of the wells are compared to each other and to the emissions of wells containing control solutions. The emission of wells with characterized compounds is correlated to the log(P) of the compounds, and/or their ability to diffuse into cell membranes, and/or the transcellular permeability, and/or the oral absorption of the compounds and/or compound ionization state, and/or the volume of distribution of the compounds and/or their distribution into different tissues and/or their partitioning into cell organelles, and these correlations are used to gauge or estimate or predict the log(P) and/or the transcellular permeability and/or oral absorption of compounds and/or compound ionization state, and/or the volume of distribution of the compounds and/or their distribution into different tissues and/or their partitioning into cell organelles for which these are unknown. The arrays may contain non-diacetylene species including, but not limited to: phospholipids, cholesterol, steroids, other lipids, proteins, cell membrane proteins, cell membrane components and fluorophores. The lipid species or other components may be synthesized or extracted from natural sources, such as cell membranes or bulk tissue. For example, the lipids and/or other components may be extracted from the cell membranes of intestinal lining cells. Lipids may be chosen to mimic the cell membranes of specific tissue types, e.g., brain, liver, fat, etc, or to mimic the membranes of various types of organisms, e.g., bacteria, yeast, etc, or to mimic cell organelle membranes. The arrays may be used over a pH range including, but not limited to, pHs of 2-10. The array solutions have been shown to be stable in storage over 12 months in some cases. The emission of the polydiacetylene arrays may drop relative to the control emission as they are exposed to compounds with increasing log(P)s and/or transcellular permeability and/or oral absorption. This decrease may be linear or have a “step-like” character with a sharp drop at a specific log(P) value. Alternatively, the emission of the arrays may rise relative to a reference as the compound log(P) values increase. The change in emission of the arrays appears to be dependent upon formulation; a compound could be screened with multiple array formulations to improve the precision of the estimation of the log(P) and/or their ability to diffuse into cell membranes and/or transcellular permeability and/or oral absorption, and/or compound ionization state, and/or the volume of distribution of the compounds and/or their distribution into different tissues and/or their partitioning into cell organelles. Data analysis can be further refined by comparison of results for sets of compounds with structural and/or functional similarity. Analysis of the kinetic changes of the emission of arrays exposed to different compounds can also allow estimation of the log(P) and/or their ability to diffuse into cell membranes and/or transcellular permeability and/or oral absorption and/or compound ionization state, and/or the volume of distribution of the compounds and/or their distribution into different tissues and/or their partitioning into cell organelles. The emission or kinetic data gathered from the testing methods of the present disclosure can be analyzed alone, or can be integrated with computer or other models to predict physical properties of the test compounds of biological relevance. For example, data analysis can be enriched by adding information on molecular properties of the compounds, such as molecular weight, number of hydrogen bond acceptors and donors etc that can be determined from the structure of the compound. Additional experimentally or predicted compound properties may also be combined with the data obtained from the proposed method. Multivariate statistical analysis or other mathematical modeling techniques may be used for prediction of compound properties.

Another embodiment of the testing method of the present disclosure involves adding test compounds in aqueous or DMSO solutions to polymerized two-dimensional arrays such as diacetylene arrays. The arrays may be on solid supports, or coated onto nanoporous filters, or free standing. The filters may be the filters of 96-well or 384-well or other well-count filter plates. The two-dimensional arrays may be arranged in a macro-array or may be in a continuous spread. The arrays may contain non-diacetylene species, including but not limited to: phospholipids, cholesterol, steroids, other lipids, proteins, cell membrane proteins, cell membrane components and fluorophores. The lipid species may be synthesized or extracted from natural sources, such as cell membranes. For example, the lipids and/or other components may be extracted from the cell membranes of intestinal lining cells. Lipids may be chosen to mimic the cell membranes of specific tissue types, e.g., brain, liver, fat, etc, or to mimic the membranes of various types of organisms, e.g., bacteria, yeast, etc, or to mimic cell organelle membranes. The arrays may be used over a pH range including, but not limited to, pHs of 2-10. The emissions of the arrays after or during exposure to the test compound solutions are measured. When the array is on a filter, in a further step the test compound solutions may be pulled or pushed through the coated filters and the emission read after that step. The emissions of arrays exposed to characterized compounds are correlated to the log(P) of the compounds, and/or their ability to diffuse into cell membranes, and/or the transcellular permeability, and/or the oral absorption of the compounds and/or compound ionization state, and/or the volume of distribution of the compounds and/or their distribution into different tissues and/or their partitioning into cell organelles; and these correlations are used to gauge or estimate or predict the log(P) and/or ability to diffuse into cell membranes and/or transcellular permeability and/or oral absorption of compounds and/or compound ionization state, and/or the volume of distribution of the compounds and/or their distribution into different tissues and/or their partitioning into cell organelles for which these are unknown. The extent of change in emission of the arrays appears to be dependent upon array formulation as well as test compound properties; a compound could be screened with multiple array formulations to improve the precision of the estimation of the log(P) and/or ability to diffuse into cell membranes and/or transcellular permeability and/or oral absorption and/or compound ionization state, and/or the volume of distribution of the compounds and/or their distribution into different tissues and/or their partitioning into cell organelles. Data analysis can be further refined by comparison of results for sets of compounds with structural and/or functional similarity. Analysis of the kinetic changes of the emission of arrays exposed to different compounds can also allow estimation of the log(P) and/or ability to diffuse into cell membranes and/or transcellular permeability and/or oral absorption and/or compound ionization state, and/or the volume of distribution of the compounds and/or their distribution into different tissues and/or their partitioning into cell organelles. The emission or kinetic data gathered from the testing methods of the present disclosure can be analyzed alone, or can be integrated with computer or other models to predict physical properties of the test compounds of biological relevance.

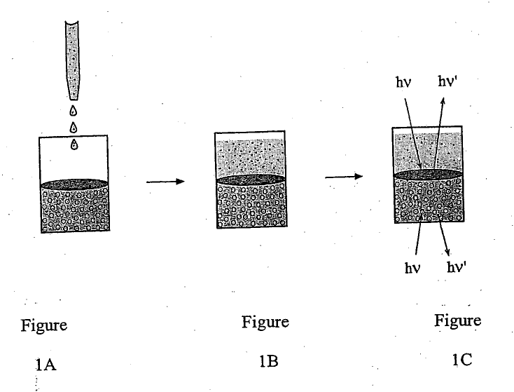

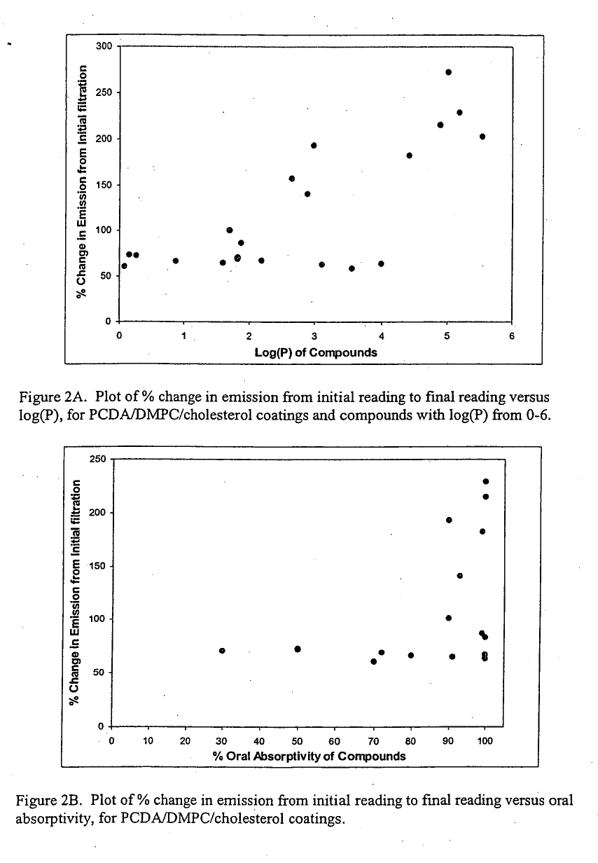

Another embodiment of the testing method of the present disclosure employs a polymerized two-dimensional diacetylene array as a thin mono, bi or multi-layer film. The passage of test compounds through this array as well as the change in emission of the array can be correlated to the absorption potential of the test compounds or to their partition coefficient. A test cell can be set up with the two-dimensional array separating a solution of three-dimensional arrays and a solution of the compound to be tested, as depicted in FIGS. 1A, 1B and 1C. FIG. 1A depicts a cell containing a 2-D array film or coating and a 3-D array solution, while FIG. 2B shows the cell with the addition of a test compound solution. The test compound partitions into and permeates the 2-D array passing into the 3-D array solution. The emissions of the 2-D and 3-D arrays are measured as illustrated in FIG. 1C, and the measured values are used to estimate the partition coefficient and/or their ability to diffuse into cell membranes and/or oral absorptivity and/or transcellular permeability of the test compound and/or compound ionization state, and/or the volume of distribution of the compounds and/or their distribution into different tissues and/or their partitioning into cell organelles. Alternatively a polymerized array supported on a nanoporous membrane could be used in lieu of the film. One example of a test cell would be a coated filtration microtiter plate well. With appropriate instrumentation, such as bottom/top reading fluorescent plate readers, it is possible to measure the emission of both the three-dimensional array solution and two-dimensional array in situ. The emission of the two- the two-dimensional and the three-dimensional arrays are measured after a set time, or monitored at intervals, and correlated to the partition coefficient, and/or their ability to diffuse into cell membranes, and/or transcellular permeability, and/or the oral absorptivity of the compound and/or compound ionization state, and/or the volume of distribution of the compounds and/or their distribution into different tissues and/or their partitioning into cell organelles.

This testing method could be used in a further extension to determine compound binding to a protein or other macromolecules in solution. Suitable proteins include, but are not limited to, blood plasma proteins. In this embodiment of the proposed disclosure, the compound is exposed to a protein and the mixture is then exposed to the polydiacetylene three-dimensional or two-dimensional array. Optionally, the fluorescence of the array is compared to that of an array exposed to the compound alone, or compared to the fluorescence of the array exposed to another appropriate reference solution, to evaluate whether the compound has been bound by the protein.

Another embodiment of the testing method of the present disclosure employs a test in which the test compound and a protein or proteins or other macromolecules are placed in solution in a test cell in which this solution is separated from a three-dimensional array solution by a membrane of appropriate pore size, for example a dialysis membrane. The membrane prevents the protein or proteins or other macromolecules and any compound that may be bound to the protein, proteins, or macromolecule from coming into contact with the array solution; whereas, unbound compound can pass through the membrane and contact the array solution. The emission of the three-dimensional arrays is measured after a set time, or monitored at intervals, to determine the binding of the test compound to the proteins or other macromolecules.

The following non-limiting examples are presented to further facilitate an understanding of the present disclosure.

In the following examples, unless otherwise stated, the diacetylene fatty acids were purchased from GFS or synthesized in-house. 10,12-Pentacosadiynoic acid (PCDA) was purchased; 6,8-docosadiynoic acid (DCDA), N-(2-Hydroxyethyl)-10,12-pentacosadiynamide (PCDA-EtOH) (Spevak et al, J. Am. Chem. Soc., 1993, 115, 1146-1147), (S)-N-(2-pentanedioic acid)-10,12-pentacosadiynamide (PCDA-Glu) (Cheng Q.; Stevens R. C., Langmuir, 1998, 14, 1974-1976) and mono 10,12-pentacosadiynyl phosphate (PCDA-PO4) (Hub H-H.; Hupfer B.; Koch H.; Ringsdorf H., Angew. Chem. Int. Ed. Engl., 1980, 19(11), 938-940) were synthesized. Acetylene compounds were purchased from GFS or Lancaster Synthesis. Reagents, anhydrous solvents, test compounds, cholesterol and buffer salts were obtained from Sigma-Aldrich and Fisher Scientific. Dimyristoyl phosphatidylcholine (DMPC), dioleoyol phosphatidylcholine (DOPC), dioleoyol phosphatidylethanolamine (DOPE), brain extract total lipids, and liver extract total lipids were purchased from Avanti Lipids. Organic fluorophores were obtained from Molecular Probes and Sigma-Aldrich; terbium and europium salts from Alfa Aesar. Solvents were obtained from Fisher Scientific in Optima grade unless otherwise specified. H2O was purified by ultra-filtration through a Millipore Milli-Q Plus system to a resistivity of 18.2 MΩ.

Probe sonication was achieved using an Imaging Products Sonic 300 V/T fitted with a micro-tip, with the power set to ˜30%. Bath sonication was achieved using a Fisher Scientific FS140H sonicator, filled with water and heated to the appropriate temperature. A Kinematica PTA10 Polytron and a Cafrano BDC 1850 Teflon/glass homogenizer were used for dispersion and homogenization steps in the preparation of lipid extracts from cells. Sorvell RC5C and Beckman L7-35 centrifuges were used for centrifugation steps. Photopolymerization was achieved using a UV-oven capable of delivering calibrated energy doses of UV light around 254 nm. Assay data were collected using both the SPEX Fluoromax-2 plate reader, and Wallach Victor2 and Victor2V readers. 384-well and 96-well black untreated polystyrene microtiter plates from Corning, and 96-well filter plates from Millipore were used in assays. 1H and 13C spectra were obtained by Acorn NMR, Livermore Calif.

Natural lipid extracts were also obtained by processing pig intestinal lining cells and Caco-2 cells using an adaptation of literature methods. Procedures were performed keeping the solutions and mixtures on ice whenever possible; centrifuges were refrigerated to 4° C. Cells were washed with 0.3M Mannitol/10 mM HEPES/10 mM TRIS buffer (pH 7.4, Buffer 1), homogenized in 0.3M Mannitol/1 mM Dithiothreitol (DTT)/1 mM TRIS/0.1 mM Phenylmethylsulfonylfluoride (pH 7.0), and the suspension centrifuged at 500 g for 5 minutes. The pellets were homogenized in 0.3 M Mannitol/1 mM DTT/1 mM TRIS (pH 7.0) and centrifuged at 500 g for 5 minutes. At this point the preparations for the two types of cells deviated.

For the pig intestinal lining cell preparation the pellets were discarded, the supernatants combined and made up in volume to 300 mL, and 3 mL of 1 mM CaCl₂ added dropwise. The mixture was stirred for 45 minutes and then centrifuged at 5,500 g for 5 minutes. The supernatants were poured off and centrifuged at 38,000 g for 40 minutes. The pellets were made up to 5 mL volume with Buffer 1 and homogenized 10 times at 2,000 RPM with a glass/Teflon homogenizer. The resuspended pellets were split into two 65 mL centrifuge tubes and diluted to full tube volume with Buffer 1. These were centrifuged at 80,000 g for 30 minutes.

For the Caco-2 cell preparation the pellets were made up to 5 mL volume with Buffer 1 and homogenized 10 times at 2,000 RPM with a glass/Teflon homogenizer. The re-suspended pellets were split into two 65 mL tubes and diluted to full tube volume with Buffer 1. These were centrifuged at 80,000 g for 30 minutes.

For both preparations the final pellets were extracted with ice-cold, Ar-sparged, 1:1 CHCl₃/MeOH with vortexing followed by filtration. The solids were then sonicated in 1:1 CHCl₃/MeOH using a bath sonicator without heating, for 5 minutes, and filtered. This step was repeated. The combined filtrates had the solvent removed by rotary evaporation under partial pressure, without any heating of the flask. 4:1 benzene/ethanol was used to azeotropically remove water. The dried solids were partially re-dissolved in 1:1 CHCI₃/MeOH at 1.4 mg/mL; undissolved solids were removed by filtration. The filtrate solvent was removed as above and the solids dissolved in 1:1 CHCl₃/MeOH to 7 mg/mL (pig intestinal cell extracts) and 4 mg/mL (Caco-2 cell extracts). The solutions were stored at −20° C.

Chloroform solutions of diacetylene surfactants were prepared by dissolving the appropriate amount of surfactant in chloroform and filtering the solution through 0.22 micron pore PTFE filters Diacetylene liposome solutions were prepared according to general methods presented in the literature (Hupfer B. et al, Chem. Phys. Lipids, 1983, 33, 355-374; Spevak et al, J. Am. Chem. Soc., 1993, 115, 1146-supplementary materials; Reichart A. et al. J. Am. Chem. Soc., 1995, 117, 829-supplementary materials) by drying organic solutions of the diacetylene surfactants together with organic solutions of any additives (e.g. phospholipids, lipids, cholesterol, and/or fluorophores), adding water or buffer to bring the combined materials to approximately 1 mM overall, using probe sonication to disperse the materials, filtering the dispersion through a 0.8 μm pore size cellulose acetate filter and chilling at 4° C. or 10° C. overnight. Coatings were prepared by using suction to filter liposome solutions through nano-porous membranes (50-200 nm), or by extruding liposome solutions through nano-porous membranes. Liposomes and coatings were polymerized using a UV (˜250 nm) cross-linking oven capable of delivering a controlled dose. Compositions are given as molar percentages unless otherwise noted. Emission excitation is at 470 nm unless otherwise noted.

Fluorophores included: 5-(((4-(4,4-difluoro-5-(2-thienyl)-4-bora-3a,4a-diaza-s-indacene-3-yl)phenoxy)acetyl)amino)pentylamine hydrochloride (1); (E,E)-3,5-bis(4-phenyl-1,3-butadienyl)-4,4-difluoro-4-bora-3a,4a-diaza-s-indacene (2); 1,1′-dioctadecyl-3,3,3′,3′-tetramethylindodicarbocyanine, 4-chlorobenzenesulfonate salt (DIC-18(5)) (3), and 6-(((4,4-difluoro-5-(2-thienyl)-4-bora-3z,4z-diaza-s-indacene-3-yl)styryloxy)acetyl) aminohexanoic6-(((4,4-difluoro-5-(2-thienyl)-4-bora-3a,4a-diaza-s-indacene-3-yl)styryloxy)acetyl)aminohexanoic acid sodium-4-sulfonate-2,3,5,6-tetrafluorophenyl ester (4).

Test compounds were taken from a set including: acebutolol, alprenolol, amiodarone, amoxicillin, ampicillin, ascorbic acid, atenolol, bifonazole, benzyl penicillin, caffeine, carbamazepine, ceftriazone, cimetidine, clofazimine, chlorpromazine, deferoxamine, desipramine, beta-estradiol, famotidine, flavone, foscarnet, fosformycin, furosemide, hydrochlorthiazide, hydrocortisone, ibuprofen, imipramine, isoniazide, kepone, ketoprofen, lactulose, mannitol, metoprolol tartrate, methotrexate, norfloxacin, ouabin, poly(ethylene oxide), pimozide, prednisone, pregnenolone, procainamide, propranolol, quinine, raffinose, ranitidine, salicylic acid, sulfasalazine, tetracycline, terbutaline, terfenadine, timolol maleate, triflupromazine, trifluoperazine. The compounds were dissolved in DMSO at 5.00 mM and further diluted with DMSO or H₂O as appropriate.

### EXAMPLE 1

Liposomes were prepared from 70% PCDA, 30% DMPC and from 70% PCDA, 30% DMPC with fluorophores 1 and 2 incorporated separately at 1 fluorophore:200 lipids, by probe sonication of the dried lipids in 2 mM HEPES buffer at pH 7.4. The solution was filtered through a 0.8 μ acetate filter, chilled at 10° C. overnight and photopolymerized with 0.2 J/cm² of UV light at about 254 nm. 4 μL of liposome solution, 32 μL of buffer (10 mM sodium phosphate, pH 6.5) and 4 μL of test compound solution (0.5 mM in DMSO) were combined in triplicate wells of a black 384 well plate. The plate was shaken for one hour and the fluorescence of the wells measured and averaged as in Example 1 and compared to the reference wells. The liposomes exposed to compounds with partition coefficients (log(P)s) of 4 or lower have emissions similar to the reference liposomes; liposomes exposed to compounds with partition coefficients above 5 have emissions that are approximately 60% of the reference emissions. The sharp “step” in the emissions around partition coefficients of 5 makes these liposomes suitable for a simple screen to see if drug candidates will have a partition coefficient above or below 5.

### EXAMPLE 2

Liposomes were prepared from 100% PCDA with fluorophores 1-3 in 2 mM HEPES at pH 6.5 and polymerized with 400 mJ/cm² UV as described above. 4 μL of liposome solution, 32 μL of buffer (10 mM sodium phosphate, pH 6.5) and 4 μL of test compound solution (0.5 mM in DMSO) were combined in triplicate wells of a black 384 well plate. The plate was shaken for one hour and the fluorescence of the wells measured and averaged as in Example 1 and compared to the reference wells. Plots of the partition coefficients of test compounds versus fluorescence of the liposomes with 1 and 2 incorporated at all three emission wavelengths showed a linear relationship with negative slopes. Liposomes with 3 incorporated showed no response to the test compounds with partition coefficients below 5.2 when compared to the reference. The emission at 675 nm rose linearly with the rise in partition coefficients above 5.2; the emissions at 642 nm and 572 nm dropped, also in a linear fashion.

### EXAMPLE 3

Liposomes were prepared from 57% DCDA, 26% DOPC, 7% DOPE, and 10% cholesterol, and from 57% DCDA, 26% DOPC, 7% DOPE, and 10% cholesterol with 2, 3, or 3 incorporated separately in 2 mM HEPES buffer at pH 7.4 and photopolymerized with 0.4 J/cm² of UV as described above. 10 μL of test compound solutions (0.5 mM in DMSO), 80 μL of buffer (2 mM HEPES at pH 6.5) and 10 μL of liposome solution were combined in triplicate samples in the wells of a black 96-well micro-titer plate. The emission of the wells at 675 nm, 642 nm and 572 nm (excitation at 470 nm) was read at 20 and 30 minutes, and averaged over triplicate wells. A roughly linear relationship is observed between the partition coefficients of the compounds and the emission above partition coefficient value of ˜5; below a partition coefficient of ˜5 the emission is more invariant.

### EXAMPLE 4

Liposomes were prepared from 44% PCDA-EtOH/26% DOPC/7% DOPE/23% cholesterol and from 44% PCDA-EtOH/26% DOPC/7% DOPE/23% cholesterol with fluorophores 2, 3, and 4 incorporated as above. The liposomes were polymerized with 0.2 J/cm2 of UV. 4 μL of liposome solution, 32 μL of buffer (10 mM sodium phosphate, pH 6.5) and 4 μL of test compound solution (0.5 mM in DMSO) were combined in triplicate wells of a black 384 well plate. The plate was shaken intermittently over one hour and the fluorescence of the wells measured and averaged as in Example 1 and compared to the reference wells. For all three emission wavelengths and for all four formulations the emissions showed a sharp drop between partition coefficients of 4 and 5, similar to that seen in Example 1.

Polymerized liposomes from 44% PCDA-EtOH/26% DOPC/7% DOPE/23% cholesterol and from 44% PCDA-EtOH/26% DOPC/7% DOPE/23% cholesterol with fluorophores 2 and 4 incorporated, were also combined with buffer and test compound solutions (0.25 mM in 5% DMSO/H₂O) in triplicate wells of a black 384 well plate as described above. The plate was shaken and the emissions read at 1 h, 3 h and 5 h as above. The emission data for the plain liposomes (without fluorophore) and the liposomes with 4 incorporated showed a roughly linear drop in emission above log(P) ˜3. The emission data for the plain liposomes also showed some correlation with the oral absorptivity of the compounds with the wells with the compound with the lowest oral absorptivity having a significantly higher emission than the other wells. These results were obtained with the test compounds at 25 μM in the assay overall, versus the 50 μM test compounds used in the first part of the example.

### EXAMPLE 5

Liposomes were prepared from 44% PCDA-Glu/26% DOPC/7% DOPE/23% cholesterol and from 44% PCDA-EtOH/26% DOPC/7% DOPE/23% cholesterol with fluorophores 2, 3, and 4 incorporated as above. The liposomes were polymerized with 0.2 J/cm² of UV. 4 μL of liposome solution, 32 μL of buffer (10 mM sodium phosphate, pH 6.5) and 4 μL of test compound solution (0.5 mM in DMSO) were combined in triplicate wells of a black 384 well plate. The plate was shaken intermittently over one hour and the fluorescence of the wells measured, averaged as in Example 1 and compared to the reference wells. For all three emission wavelengths and for all four formulations the emissions showed a sharp drop between partition coefficients of 4 and 5, similar to that seen in Example 1 and Example 4.

### EXAMPLE 6

Liposomes were prepared from 44% PCDA-Glu/26% DOPC/7% DOPE/23% cholesterol, from 44% PCDA-EtOH/26% DOPC/7% DOPE/23% cholesterol and from 44% PCDA-PO₄/26% DOPC/7% DOPE/23% cholesterol with fluorophore 4 incorporated as above. The liposomes were polymerized with 0.2 J/cm² of UV. 4 μL of liposome solution, 32 μL of buffer (10 mM sodium phosphate, pH 6.5) and 4 μL of test compound solution (0.5 mM in DMSO) were combined in triplicate wells of a black 384 well plate. A second plate was prepared with equivalent amounts using 10 mM sodium phosphate at pH 7.4. The plates were shaken for one hour at 37° C. and the emissions of the wells measured (also at 37° C.) and averaged as in Example 1. The plates were kept at 37° C. and measured again at 3 h, 5 h and 20 h. The data trends did not change significantly over time or with pH. For all three liposome formulations, at both pHs, the 572 nm emission showed a strong drop in emission between log(P) values of 4 and 5; the 642 nm and 675 nm emissions showed a smaller drop.

### EXAMPLE 7

Liposomes were prepared from 44% PCDA-Glu/26% DOPC/7% DOPE/23% cholesterol with fluorophore 2 incorporated (1) and from 44% PCDA-EtOH/26% DOPC/7% DOPE/23% cholesterol with fluorophore 4 incorporated (2) as above. A 96-well filter plate with 0.05μ pore mixed cellulose ester (MCE) filters from Millipore was pre-wetted by vacuum pulling 100-300 uL of deionized water through the filters. Three columns were charged with 75 μL of (1) per well; three columns were charged with 75 μL of (2) per well and two columns were charged with 75 μL of (1) and 75 μL of (2). The solutions were pulled through the filters under negative pressure using a Millipore vacuum manifold designed for plate filtering and a water aspirator. The plates were chilled at 4° C. for at least 2 hour and then exposed to 20 mJ/cm² to polymerize the coatings, which formed even blue coatings. The plate was stored at 4° C. between preparation and use.

The drip directors on the plate bottom were sealed with water-proof tape, and 90 μL of 10 mM sodium phosphate, pH 6.5, were added to each well. After ˜10 m the emission of the wells at 675 nm, 642 nm and 572 nm were measured (ex=470 nm). 10 μL of seven test compounds at 0.5 mM in DMSO were added to triplicate wells (duplicate wells for wells coated with (1) and (2) together). The plate was shaken at RT for one hour and the emission of the wells read. The plate was read again at 2.25 h, 4 h, and 20 h. The tape was removed, the plate was then placed back on the Millipore vacuum manifold and negative pressure down to 30 Torr was applied. The solutions filtered very slowly; some wells showed no appreciable filtration. The plate was read again. The percent change in emission of the wells exposed to test compounds from the initial emission values in buffer was calculated and averaged over triplicate or duplicate wells. For the coatings made from (1) and (2) together the percent change showed a roughly linear relationship with log(P) with a negative slope; for the coatings made from (1) and (2) separately below log(P)˜2 the percent change is fairly invariant and above log(P) of 2 there is a roughly linear decrease as the log(P) increases. The coatings from (1) and (2) together also show a trend of percent emission change with oral absorption; the compounds with oral absorptions of 90% or higher had percent emission changes of −28% to −39% while the compound with oral absorption of 1% had a percent emission change of −14%, all at 675 nm. Similar trends were seen for emissions at 642 nm and 572 nm. The trends discussed above were seen at all time points and after the filtration step. The coatings made from (2) also showed a trend of percent emission change with Caco-2 permeability with a sharp drop in percent emission change between Caco-2 permeability of 21×10⁻⁶ and 1×10⁻⁶ cm/sec.

### EXAMPLE 8

Liposomes were prepared from 44% PCDA-Glu/33%DMPC/23% cholesterol in 2 mM HEPES at pH 7.4 as described above. Coatings were deposited on a pre-wetted 96-well filter plate onto 0.1μ MCE filters at 350 Torr, as described in Example 7 above, and photopolymerized with 10 mJ/cm² of UV to form deep blue-purple coatings.

The coatings were exposed to 100 μL of 10 mM sodium phosphate at pH 6.5 (buffer) and shaken for 20 minutes. The buffer was then vacuum filtered through the wells at 300 Torr and the emission measured at 545 nm (initial emission). Diluted test compounds were prepared by combining 30 uL of 5 mM test compound solution in DMSO with 2.97 mL of buffer. 100 μL of this was added to the wells and the plates shaken for 4 hours. The solutions were then filtered through the wells, at 300 Torr, and the emission measured again (final emission). FIG. 2A shows the change between the initial and final emissions plotted versus the log(P) of the compounds for compounds with log(P) between 0-6, and FIG. 2B shows the change between the initial and final emissions plotted versus the oral absorptivity of the compounds.

### EXAMPLE 9

Liposomes were prepared from PCDA, PCDA-EtOH and PCDA-Glu with brain total lipid extracts or liver total lipid extracts, using a total of 8.8×10-7 moles diacetylene surfactant and 6.9×10-4 grams of extracted lipids, in 2 mM HEPES at pH 7.4 for PCDA and PCDA-Glu based liposomes, and 2 mM HEPES at pH 6.5 for PCDA-EtOH based liposomes, as described in previous examples. The liposomes were polymerized with 0.2 J/cm² of UV; the PCDA/liver lipids liposomes did not polymerize and were discarded. 4 μL of polymerized liposome solution, 32 μL of buffer (10 mM sodium phosphate, pH 6.5) and 4 μL of test compound solution (at 0.05 mM in DMSO) were combined in triplicate wells of a black 384 well plate. The plate was shaken intermittently over one hour and the fluorescence of the wells measured and averaged as in Example 1 and compared to the reference wells. The trends of change in emission relative to the reference versus log(P) were similar to those seen with the liposomes with DOPC/DOPE/cholesterol incorporated, as described in previous examples.

### EXAMPLE 10

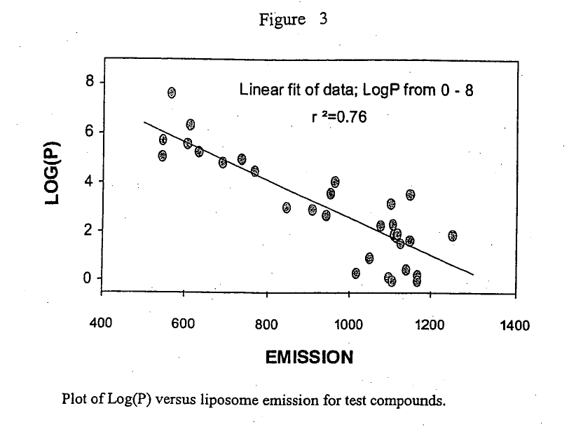

Liposomes were prepared from PCDA-EtOH and PCDA-Glu with lipids from Caco-2 cells and cholesterol, using 1.75 μmoles of diacetylene, 9.6×10-4 g lipid and 4.48 μmoles cholesterol, in 2 mM HEPES at pH 6.5 and 7.4 respectively. The liposomes were polymerized with 0.2 J/cm2 of UV, in a chilled 24-well plate on ice, and then heated at 70° C. for 5 minutes under an argon stream. 1.2 mL of liposomes were diluted with 10.4 mL of buffer (10 mM sodium phosphate/150 mM sodium chloride, pH 6.5). 58 μL of diluted liposomes and 2 μL of test compound solution (5 mM in DMSO) were combined in the wells of a black 384-well plate and the fluorescence at 545 nm was read every three minutes with shaking between readings. A plot of the 15 minute data of emission vs log (P) of the compounds for log (P) from 0-8 was fitted to a line with a R² value of 0.76, as shown in FIG. 3.

### EXAMPLE 11

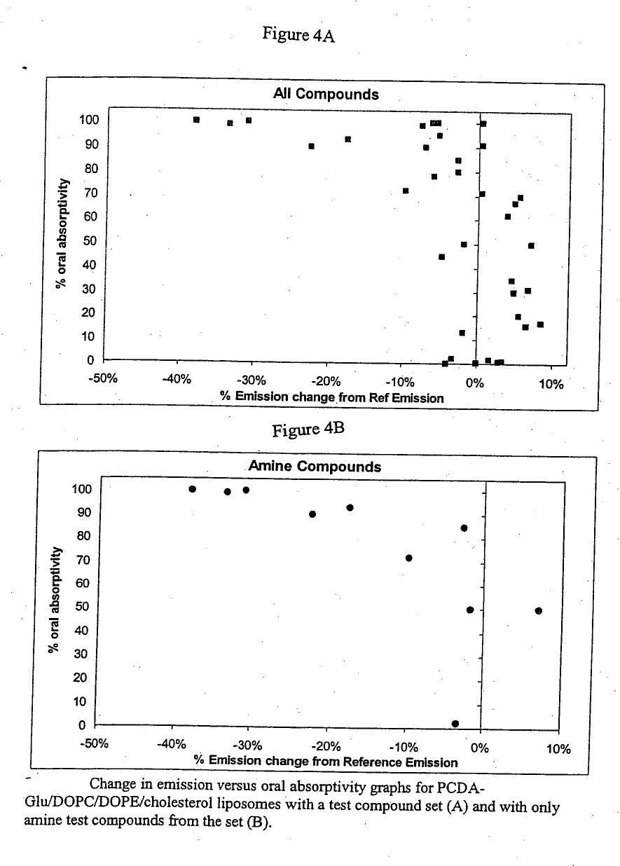

Liposomes were prepared from 44% PCDA-Glu/26% DOPC/7% DOPE/23% cholesterol in 2 mM HEPES at pH 7.4, polymerized with 0.2 J/cm² of UV as described for Example 6, then heated at 70° C. for 5 minutes under a stream of argon. 1.154 mL of polymerized liposomes were combined with 8.840 mL of 10 mM sodium phosphate/150 mM sodium chloride buffer at pH 6.5. 52 μL of the diluted liposomes were combined with 8 uL of test compound solutions (0.25 mM in 5% DMSO/H₂O) in the wells of a 384-well black plate. The plate was shaken and fluorescence emission readings at 545 nm were taken at intervals. FIGS. 4A and 4B show a plot of the change in the emission versus a reference solution versus oral absorptivity for all the compounds (FIG. 4A), and for only the amine compounds (FIG. 4B), at four hours.

### EXAMPLE 12

Liposomes were prepared from PDA-EtOH (34.7 weight %), lipids extracted from swine duodenum mucosa cells (57.8 weight %), and cholesterol (8.5 weight %), in 2 mM HEPES at pH 6.5, polymerized with 0.2 J/cm² of UV, and heated at 70° C. for 5 minutes as described above. 6 μL of liposome solution, 8 μL of test compound solutions (0.25 mM in 5% DMSO/H₂O), and 46 μL of 10 mM sodium phosphate buffer at pH 6.5 were combined in the wells of a black 384-well plate. The plate was shaken and the emission at 675 nm, 642 nm and 572 nm were read at intervals over 99 hours; in between readings the plate was covered with a sealed cover. Comparison of plots of change in emission from the reference versus % oral absorptivity taken at different time points showed that measuring the emission any time from 15 minutes to 51 hours gave equivalent results. The plots showed a drop in the emission versus the reference for compounds with oral absorptions >80%.

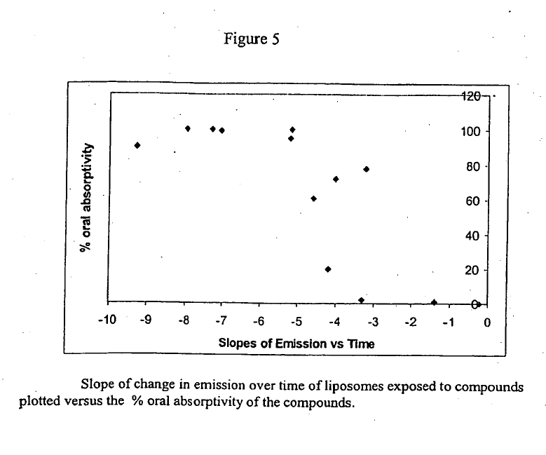

The slopes of the change in the emission versus time were calculated using data collected in the first hour. The slopes were plotted against the % oral absorption of the compounds as shown in FIG. 5. This demonstrates an alternative way of processing the data for oral absorption prediction.

### EXAMPLE 13

Liposomes were prepared from 44% PCDA-EtOH/26% DOPC/7% DOPE/23% cholesterol, as described in Example 4, with fluorophore 2 incorporated, and heated for 5 minutes at 70° C. 85 μL of 0.278 mg/mL solution of human serum albumin (HSA) in 10 mM sodium phosphate, pH 7.4, were incubated at room temperature with 5 μL each of two test compounds (2 mM in DMSO) and also with 5 μL DMSO as a reference, for 1.25 hours in the wells of a 384-well black plate, with shaking. 10 μL of the liposome solution were added to the mixed solutions, the plate was shaken further, and the emission read at intervals. One test compound had a relatively high affinity for HSA, and the averaged emissions of wells with HSA and the test compound together, and with the HSA alone, were similar, and differed from the averaged emissions of wells with the test compound alone. These results suggested that the test compound was bound to the HSA and not free to interact with the liposomes. The other compound had a low affinity for HSA, and the averaged emissions of the wells with the test compound and HSA and of the averaged emissions of wells with the test compound alone were similar. These results suggested that the test compound was free to interact with the liposomes.

The foregoing description illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the disclosure and to enable others skilled in the art to utilize it in such, or other, embodiments and with the various modifications required by the particular applications or uses thereof. Accordingly, the description is not intended to limit the invention to the form disclosed herein. Also, it is intended that the appended claims be construed to include alternative embodiments.

All publications and patent applications cited in this specification are herein incorporated by reference, and for any and all purposes, as if each individual publication or patent application were specifically and individually indicated to be incorporated by reference. In the case of inconsistencies the present disclosure will prevail.

What is claimed as new and desired to be protected by Letters Patent of the United States is:

1. A method for evaluating at least one of the ionization state of a compound, the volume of distribution of a compound, the distribution of a compound into different tissues, the ability of a compound to diffuse into cell membranes and the partitioning of a compound into cell organelles, which comprises exposing a three-dimensional array comprising a polydiacetylene backbone or a two-dimensional array comprising a polydiacetylene backbone, or both, to the compound to be evaluated; and
measuring the effect on the array by detecting the change in fluorescence or phosphorescence.

2. The method of claim 1 wherein the array comprises a three-dimensional array in the form of a solution of liposomes or tubules or ribbons or fibers.

3. The method of claim 1 wherein the polydiacetylene of the array is in the fluorescent or phosphorescent form, and the decrease in fluorescence or phosphorescence is measured.

4. The method of claim 1 wherein the increase in fluorescence or phosphorescence is measured.

5. The method of any one of claims 1 to 4 wherein the three-dimensional array or a two-dimensional array further comprises a fluorophore and wherein the change in fluorescence or phosphorescence of the polydiacetylene array is monitored.

6. The method of any one of claims 1 to 4 wherein the three-dimensional array or a two-dimensional array further comprises a fluorophore or phosphor and wherein the change in fluorescence of the fluorophore or phosphorescence of the phosphor is monitored.

7. The method of claim 1 wherein array does not contain a further fluorophore or further phosphor.

8. The method of claim 1 wherein the change in fluorescence is detected by exposure to light having wavelengths below 550 nm and measurement of the emission.

9. The method of claim 1 wherein the change in fluorescence is detected by exposure to light having wavelengths between 450 and 500 nm and measurement of the emission.

10. The method of claim 1 wherein the array comprises a two-dimensional array of a polydiacetylene backbone coated onto a solid support.

11. The method of claim 10 wherein the solid support is a porous membrane.

12. The method of claim 1 wherein the array is a two-dimensional array of a polydiacetylene backbone and is unsupported.

13. The method of claim 1 wherein the array comprises a two-dimensional array of a polydiacetylene backbone and which further comprises providing a filter or flow cell containing the array; and passing a solution of the compound through the filter or flow cell before or during the detecting.

14. The method of claim 1 wherein the array is a two-dimensional array of a polydiacetylene backbone located on a non-porous support.

15. The method of claim 1 which further comprises comparing the change in fluorescence or phosphorescence from exposure of the array of the kind being used to a reference.
